(19) United States
(12) Patent Application Publication
(10) Pub. No.: US 2015/0073215 A1
(43) Pub. Date: Mar. 12, 2015

(54) ENDOSCOPE OVERTUBE

(71) Applicant: OSAKA UNIVERSITY, Osaka (JP)

(72) Inventor: Kiyokazu Nakajima, Osaka (JP)

(21) Appl. No.: 14/387,854

(22) PCT Filed: Mar. 26, 2013
(86) PCT No.: PCT/JP2013/058677

§ 371 Date: Sep. 25, 2014

(30) Foreign Application Priority Data
Mar. 27, 2012 (JP) 2012-070724

(51) Int. Cl. A61B 1/00

(52) U.S. Cl. CPC A61B 1/00135
USPC 600/114

(57) ABSTRACT

Disclosed is an endoscope overtube such that friction during turning can be reduced. The endoscope overtube of the present invention has a first insertion passage into which an endoscope can be inserted, and has a second insertion passage into which a treatment instrument can be inserted, in a wall constituting the first insertion passage, wherein the second insertion passage has, on a side face thereof, an opening which passes through a surface of the overtube.

### TECHNICAL FIELD

The present invention relates to an endoscope overtube. More specifically, the invention relates to an endoscope overtube whose friction during turning can be reduced.

### BACKGROUND ART

In general, an endoscope is provided, at its tip portion, with an observation optical system, an illumination optical system for illuminating an affected area, an air supply and water supply channel, a treatment instrument channel, and so forth. Using the endoscope, it is possible to perform various maneuvers such as specimen retrieval, removal of a foreign body, hemostasis, resection of a tumor, and morcellation of a gallstone by introducing a treatment instrument such as a forceps from the treatment instrument channel while observing an affected area in a lumen such as stomach from a video camera portion. Overtubes for assisting insertion of an endoscope or for simultaneous insertion of a plurality of endoscopes or treatment instruments are also known (for example, Patent Documents 1 and 2).

In recent years, new medical treatment techniques using a flexible endoscope, including, for example, endoscopic mucosal resection (EMR), endoscopic submucosal dissection (ESD), endoscopic aspiration mucosectomy (EAM), and endoscopic variceal ligation (EVL) have been developed and are receiving attention in the field of digestive system diseases. Recently, clinical introduction of the surgery called Natural Orifice Translumenal Endoscopic Surgery (NOTES) (surgery by which a flexible endoscope is inserted from mouth or anus into a lumen and is caused to reach the inside of a body cavity through the incision of the wall of the lumen) has also been started. These medical treatments using a flexible endoscope do not theoretically require incision of a body surface, and are therefore expected to become one of the pillars of medical care in the 21st century as patient friendly, minimally invasive medical treatment techniques.

To perform a treatment endoscopically, a long and flexible treatment instrument is inserted through an insertion passage (treatment instrument channel) provided in an endoscope and/or an overtube. The treatment instrument inserted through the treatment instrument channel of the endoscope can be freely manipulated in the axial direction, but its manipulation in the lateral direction is restricted. Meanwhile, the treatment instrument inserted through the treatment instrument channel of the overtube can be also manipulated in the lateral direction with the viewing field of the endoscope held constant, by turning the overtube itself, and, thus, the operation of applying tension (traction) to a tissue to be treated, in a wedge resection of the intestinal lining/mucosa or the like, can be easily performed, and an increased variety of surgery maneuvers can be realized (Patent Document 2).

In general, a lubricant is applied in advance to the surface of the endoscope or the overtube before insertion such that the endoscope or the overtube can be easily turned after insertion. However, during surgery, this lubricant may flow out to be depleted, and, as a result, friction with the inside of the body may increase and the turning may be difficult. In this case, it is necessary to pull out the overtube, additionally apply the lubricant to its surface, and then re-insert the overtube, which doubles the risk of patient discomfort, mucosal damage, and the like at the time of insertion.

### PRIOR ART DOCUMENTS

### Patent Documents

Patent Document 1: Japanese Laid-Open Patent Publication No. 2008-125819
Patent Document 2: WO2011/004820

### SUMMARY OF INVENTION

### Problems to be Solved by the Invention

It is an object of the present invention to provide an endoscope overtube such that friction during turning can be reduced.

### Means for Solving the Problems

The present inventors accomplished the present invention on the basis of the finding that, when an opening which passes through the surface of an endoscope overtube is provided on a side face of an insertion passage formed in the wall of the overtube, friction of the overtube during turning can be reduced, by merely supplying a lubricant via the insertion passage to the surface of the overtube, without pulling out the overtube.

The present invention provides an endoscope overtube having a first insertion passage into which an endoscope can be inserted, including a second insertion passage into which a treatment instrument can be inserted, in a wall constituting the first insertion passage, wherein the second insertion passage has, on a side face thereof, an opening which passes through a surface of the overtube.

In one embodiment, a diameter of the opening is smaller than a diameter of the second insertion passage.

In one embodiment, the number of the opening is 2 to 5 along a major axis direction.

In one embodiment, diameters of the openings increase from a proximal end toward a distal end of the second insertion passage.

In one embodiment, intervals between the openings, and between the openings and a proximal end and a distal end of the second insertion passage are equal.

### Effects of Invention

The endoscope overtube of the present invention is characterized in that (1) a treatment instrument insertion passage is provided in the wall of a conventional endoscope overtube and in that (2) the treatment instrument insertion passage of the overtube has, on a side face thereof, an opening which passes through the surface of the overtube. Accordingly, when a lubricant is supplied to the treatment instrument insertion passage of the overtube from the proximal end side, the lubricant reaches the surface of the overtube via the opening on the side face of the insertion passage, and, thus, the overtube can be favorably turned by reducing friction during the turning. Furthermore, when a treatment instrument is inserted and manipulated through the insertion passage to which the lubricant has been supplied, the lubricant is continuously supplied to the surface of the overtube via the insertion passage, and, thus, the friction of the overtube during turning can be reduced for a long period of time. Accordingly, it is possible to reduce the number of times to interrupt the surgery, and pull out and then re-insert the overtube for the purpose of additional application of the lubricant to the surface of the overtube. As a result, the risk of patient discomfort, mucosal damage, and the like, caused by repeatedly pulling out and re-inserting the overtube, can be reduced, and the treatment instrument can be favorably manipulated together with the endoscope, so that the time and effort required for the treatment can be reduced.

### BRIEF DESCRIPTION OF DRAWINGS

FIG. 1 is a schematic view of an overtube, illustrating an exemplary endoscope overtube of the present invention.

FIG. 2 is a partially enlarged cross-sectional view in the major axis direction of the endoscope overtube of the present invention shown in FIG. 1.

FIG. 3 is a view illustrating an example of a use state of the endoscope overtube of the present invention, showing an enlarged view of a distal end portion of the overtube illustrating a state in which an endoscope is inserted through a first insertion passage, an electrocautery is inserted through a treatment instrument channel of the endoscope, and a grasping forceps is inserted through a second insertion passage of the overtube.

FIG. 4 is a schematic view of an overtube, illustrating another exemplary endoscope overtube of the present invention.

### MODE FOR CARRYING OUT THE INVENTION

In the present invention, an “endoscope” refers to a medical flexible endoscope unless otherwise stated. Such flexible endoscopes are formed of a flexible material, and can be classified as those using glass fiber and those using a CCD, as the optical system contained therein. Generally, a light source is provided in a control device outside the body, and guides light with optical fiber and irradiates the light from its tip portion. Another type of the endoscope contains an LED at its tip. In general, an endoscope includes a path (sub-lumen or channel) different from that of the optical system, thereby enabling local irrigation, injection of a gas or a liquid, spraying of medicine, suction, treatments using dedicated treatment instruments (devices), and so forth. In addition, the orientation of the tip of the endoscope can be freely changed by an operation at hand.

The size of the endoscope used in the present invention can be appropriately selected according to the hollow organ of interest. Examples of arbitrary hollow organs of interest include esophagus, stomach, small intestine, colon and rectum, vagina, and bladder.

As used herein, “proximal” refers to a portion of an instrument and an apparatus that is closer to the operator, and “distal” refers to a portion of the instrument and the apparatus that is farther from the operator. Further, “major axis direction” refers to a direction of the axial center along the longitudinal direction of the overtube.

The endoscope overtube of the present invention has a first insertion passage into which an endoscope can be inserted, and further has a second insertion passage into which a treatment instrument can be inserted, in a wall constituting the first insertion passage, wherein the second insertion passage has, on a side face thereof, an opening which passes through the surface of the overtube.

The material of the overtube is a material commonly used for a medical instrument, and is required to have flexibility, less friction (lubricity), strength, column stiffness, and so forth. Examples of polymers that can be used for such a medical instrument include flexible resins such as polyvinyl chloride, polyethylene, polyester, polyurethane, and polyamide. Polyvinyl chloride is preferable in terms of lesser friction. Furthermore, the overtube may be configured by a combination of a plurality of parts. In this case, the constituent parts may be formed of the same flexible resin, or a plurality of flexible resins.

The endoscope overtube of the present invention has a substantially cylindrical shape, for example. Alternatively, for example, when the endoscope is intended for use for surgery of stomach and esophagus, it may have a form that is curved into an arched shape for facilitating its insertion from the oral region to the esophagus of a subject. There is no particular limitation on the outer diameter of the endoscope overtube of the present invention. The outer diameter may be a size that does not cause an excessive expansion of a lumen into which the overtube is to be inserted, preferably 20 mm or less, more preferably 18 mm or less, even more preferably 15 to 18 mm.

The diameter of the first insertion passage (the inner diameter of the first insertion passage) of the endoscope overtube of the present invention may be any size that allows insertion of an endoscope. Since endoscopes having a very small diameter of 5 mm exist, the inner diameter of the first insertion passage is preferably 5 mm or greater.

In the endoscope overtube of the present invention, the wall constituting the first insertion passage includes the second insertion passage, and therefore the wall thickness in the vicinity of the first insertion passage around where at least the second insertion passage runs through in the cross-sectional direction may have a thickness greater than that of conventional overtubes. There is no particular limitation on the wall thickness as long as it is a thickness that allows formation of the second insertion passage for the treatment instrument and it allows formation of the first insertion passage for the endoscope. The wall thickness may be appropriately decided according to the outer diameter of the overtube and the inner diameter of the first insertion passage as well as the shape and the inner diameter of the second insertion passage, which will be described later. For example, when the average outer diameter of the overtube is 18 mm and the inner diameter of the first insertion passage is 12 mm, the average wall thickness of the overtube is 3 mm. There is absolutely no limitation on the average wall thickness that can be applied in the present invention because it changes, for example, according to the outer diameter of the overtube, the inner diameter of the first insertion passage, and the inner diameter of the second insertion passage, but it is preferably 2 mm or greater, more preferably 3 mm or greater, and is preferably 5 mm or less, more preferably 4 mm or less. Alternatively, there is no particular limitation on the diameter of the second insertion passage (the inner diameter of the second insertion passage) of the endoscope overtube of the present invention as long as it is a size that allows insertion of a treatment instrument intended for use, but it is preferably 2 mm to 3.5 mm, more preferably 2.8 mm to 3.2 mm.

In this manner, the shape and the size of the second insertion passage of the endoscope overtube of the present invention may be appropriately decided in consideration of treatment instruments that are commonly used in the art. The second insertion passage of the overtube is a lumen that is independent of the first insertion passage. The second insertion passage can be appropriately used not only for insertion of the treatment instrument, but also for air supply, water supply, smoke evacuation, insertion of an auxiliary treatment instrument, insertion of a second endoscope, and so forth.

The major axis direction of the second insertion passage of the endoscope overtube of the present invention may be the same as the major axis direction of the first insertion passage (i.e., the second insertion passage may extend substantially parallel to the first insertion passage extending along the major axis direction of the overtube), or may be different therefrom (i.e., the second insertion passage may extend in the form of a helix along the major axis direction of the overtube, around the first insertion passage extending along the major axis direction of the overtube), at a distal end of the second insertion passage.

For example, a single second insertion passage is provided in the overtube of the present invention. Where necessary, two or more second insertion passages may be provided independently of each other. When two or more second insertion passages are present, the major axis directions of the second insertion passages at the distal end of the overtube may be the same (i.e., may be parallel to each other), or may be different from each other.

The proximal end face and the distal end face of the wall constituting the first insertion passage of the endoscope overtube of the present invention have openings of the second insertion passage. Furthermore, the second insertion passage has, on the side face thereof, an opening which passes through the surface of the overtube. There is no particular limitation on the shape of the opening. There is no particular limitation on the diameter of the opening, but it is preferably smaller than the diameter of the second insertion passage. If the diameter of the opening is the same as or greater than the diameter of the second insertion passage, there is a risk that the treatment instrument inserted into the second insertion passage may be mistakenly protruded from the opening into the body. There is no particular limitation on the number of openings provided on the side face, but it is preferably 2 to 5 openings along the major axis direction. With a plurality of openings, the diameters of the openings more preferably increase from the proximal end toward the distal end of the second insertion passage. In this case, when a lubricant is supplied from the proximal end of the second insertion passage, the amounts of lubricant supplied from the respective openings to the surface of the overtube can be made the same. There is no particular limitation on the intervals between the openings, and between the openings and the proximal end and the distal end of the second insertion passage because they affect, for example, the length in the major axis direction of the overtube and the like, but they are preferably equal intervals. In this case, the lubricant can be constantly supplied from each opening to the surface of the overtube.

In the endoscope overtube of the present invention, the endoscope is inserted from the proximal end of the first insertion passage, and is protruded from the distal end thereof. For example, the treatment instrument is inserted from the proximal end of the second insertion passage, and is protruded from the distal end thereof. The surfaces of the first and second insertion passages may be coated with a lubricity enhancing material in order to allow the endoscope and the treatment instrument to be advanced and retracted smoothly. Preferably, the proximal end of the overtube is provided with a base end portion made of a resin that is harder than the above-described flexible resin in order to facilitate the insertion manipulation of instruments such as the endoscope and the treatment instrument. For example, a grip made of a rigid resin such as an ABS resin may be provided at the base end portion.

According to the endoscope overtube of the present invention, when a lubricant is supplied to the second insertion passage from the proximal end side, the lubricant reaches the surface of the overtube via the opening provided on the side face of the second insertion passage, and, thus, the overtube can be favorably turned by reducing friction during the turning. Furthermore, when the treatment instrument is inserted and manipulated through the second insertion passage to which the lubricant has been supplied, the lubricant is continuously supplied to the surface of the overtube via the second insertion passage, and, thus, the friction of the overtube during turning can be reduced for a long period of time. Accordingly, it is possible to avoid the necessity to interrupt the surgery, pull out the overtube, additionally apply the lubricant to its surface, and then insert the overtube again.

There is no particular limitation on the lubricant, and examples thereof include Xylocaine (registered trademark) jelly (AstraZeneca K.K.), K-Y (registered trademark) lubricating jelly (Johnson & Johnson), Through Pro (registered trademark) jelly (Kaigen Pharma Co., Ltd.), and Endolubri (registered trademark) (Olympus Medical Systems Corp.).

According to the present invention, there is no particular limitation on the treatment instrument inserted through the treatment instrument channel of the endoscope inserted through the first insertion passage, and examples thereof include treatment instruments intended for use for incision, coagulation, hemostasis, ablation, morcellation, ligation, cutting and suturing, dissection, and the like.

According to the present invention, there is no particular limitation on the treatment instrument inserted through the second insertion passage, and examples thereof include a grasping forceps and a retractor.

According to the present invention, the treatment instrument inserted through the second insertion passage of the overtube can be manipulated also in the lateral direction by turning the endoscope overtube itself without turning the endoscope (with the viewing field of the endoscope held constant), thus enabling an increased variety of surgery maneuvers such as wedge resection of the intestinal lining/mucosa to be realized.

### Examples

Hereinafter, the present invention will be described in detail with reference to the drawings.

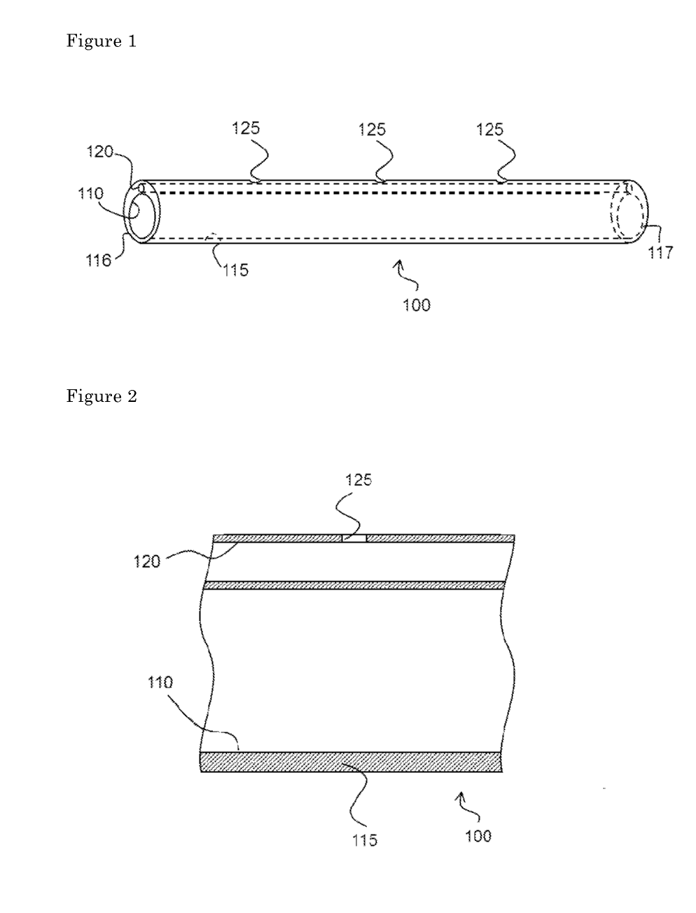

FIG. 1 is a perspective schematic view of the structure of an endoscope overtube 100 of the present invention. The endoscope overtube 100 of the present invention has a first insertion passage 110 into which an endoscope (not shown) can be inserted, and has a second insertion passage 120 into which a treatment instrument (not shown) can be inserted, in a wall 115 constituting the first insertion passage 110. In FIG. 1, the major axis direction of the second insertion passage 120 is the same as the major axis direction of the first insertion passage 110 at the distal end of the second insertion passage 120. That is to say, in the major axis direction of the overtube 100 of the present invention shown in FIG. 1, the first insertion passage 110 and the second insertion passage 120 are arranged substantially parallel to each other.

As shown in FIG. 1, the second insertion passage 120 has a plurality of openings at a distal end face 116 and a proximal end face 117 of the wall 115 constituting the first insertion passage 110. In FIG. 1, there are three openings 125 which pass through the surface of the overtube along the major axis direction on the side face, and the intervals between the openings 125, and between the openings 125 and the proximal end 117 and the distal end 116 of the second insertion passage 120 are equal.

FIG. 2 is a partially enlarged schematic view of a cross-section in the axial direction, showing the structure of the endoscope overtube 100 of the present invention. The first insertion passage 110 is disposed substantially parallel along the major axis direction of the overtube 100. In a similar manner, the second insertion passage 100 is disposed substantially parallel to the first insertion passage 110 along the major axis direction of the overtube 100. The second insertion passage 120 has, on its side face (the wall toward the overtube surface), an opening 125 which passes through the surface of the overtube.

Hereinafter, the functions of the opening 125 shown in FIG. 2 will be described. In FIG. 2, a treatment instrument is inserted and a lubricant oil is supplied, from the proximal end (on the right side in FIG. 2) of the overtube, into the second insertion passage 120 of the overtube. When the lubricant oil passes through the opening 125 provided at the second insertion passage 120, some amount of the lubricant oil flows out to the outside via the opening 125. As a result, part of the lubricant oil that has flowed out of the opening 125 is diffused to a gap between the overtube and patient's tissues in the vicinity of a position where the lubricant oil has flowed out, allowing the overtube and the tissues in that gap to be lubricated. Meanwhile, most of the lubricant oil that has not passed through the opening 125 passes through the second insertion passage 120 toward the distal end of the overtube. In this manner, the lubricant oil flows through the second insertion passage 120, also allowing to lubricant the movement of the treatment instrument within the inner wall of the second insertion passage 120.

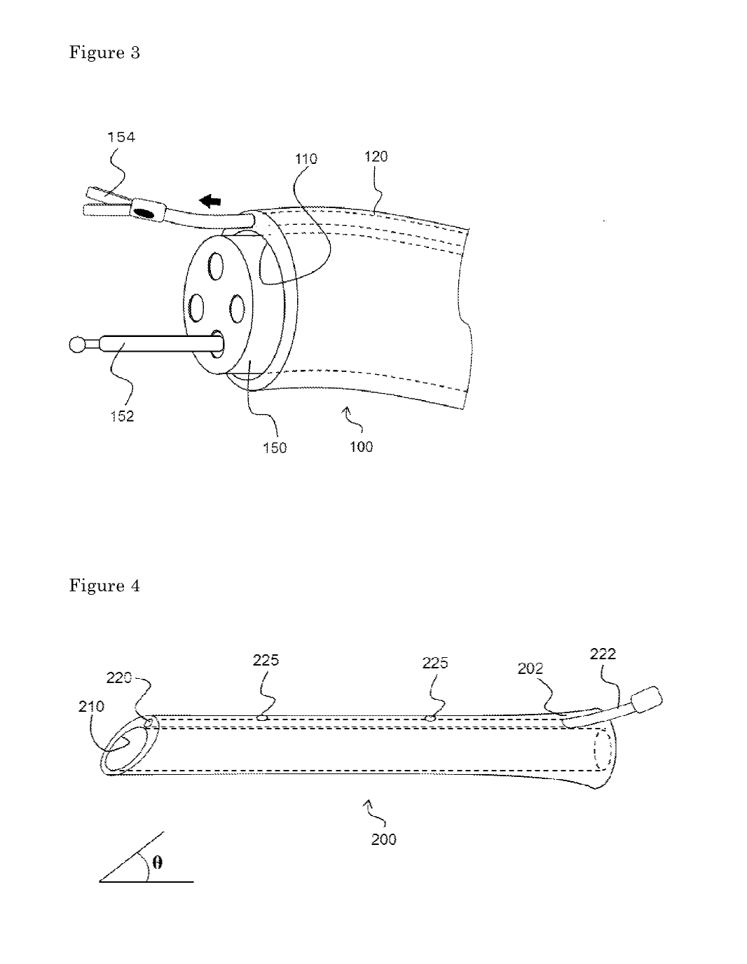

FIG. 3 is a view illustrating an example of a use state of the endoscope overtube 100 of the present invention, schematically showing the distal end of the overtube.

According to the overtube 100 of the present invention, for example, an endoscope 150 is inserted through the first insertion passage 110, an electrocautery 152 is inserted through the treatment instrument channel of the endoscope 150, and a grasping forceps 154 is inserted through the second insertion passage 120 of the overtube 100. As shown in FIG. 3, they can be protruded from the distal end of the overtube 100.

According to the present invention, for example, on endoscope surgery in a gastrointestinal tract, the grasping forceps 154 protruded from the second insertion passage 120 at the distal end of the overtube 100 grasps the surface of the gastrointestinal tract. Thus, appropriate tension (traction) can be applied to the surface of the gastrointestinal tract via the grasping forceps 154, by turning the overtube 100 itself without turning the endoscope 150 (with the viewing field of the endoscope held constant). In this state, when the electrocautery 152 or the like protruded from the endoscope 150 is brought to a desired position on the surface of the gastrointestinal tract, treatment such as resection of a tumor can be easily performed.

FIG. 4 is a schematic view of an overtube, illustrating another exemplary endoscope overtube of the present invention.

As shown in FIG. 4, an endoscope overtube 200 of the present invention is designed such that the outer diameter and the wall thickness of a tube body 202 are somewhat larger on the proximal end side than on the distal end side, in order to improve the operability in the turning. The overtube 200 is provided with, substantially parallel along the major axis direction thereof, a first insertion passage 210 for the insertion of an endoscope (not shown). The overtube 200 has, substantially parallel to the major axis direction thereof and the first insertion passage 210, a second insertion passage 220 into which a treatment instrument (not shown) can be inserted. According to the overtube 200 of the present invention, an end portion 222 of the second insertion passage 220 is protruded to the outside of the tube body 202 on the proximal end side, in order to further improve the operability of the treatment instrument inserted into the second insertion passage 220. Furthermore, the second insertion passage 120 is provided with openings 225 preferably arranged at substantially equal intervals, as in the overtube 100 of the present invention described above.

As shown in FIG. 4, the distal end of the endoscope overtube 200 of the present invention may be inclined by a predetermined angle (θ) relative to the tube body 202. There is no particular limitation on the inclination angle θ, and examples thereof include angles from 30° to 90°.

### INDUSTRIAL APPLICABILITY

The endoscope overtube of the present invention does not require an endoscope or a treatment instrument that have a special structure, and can be used together with a commonly used endoscope and a treatment instrument with a simple structure. With the use of the endoscope overtube of the present invention, the operability and safety are dramatically improved for technically difficult maneuvers such as EMR, ESD, EAM, EVL, and NOTES. Therefore, the invention is particularly useful for surgical resection of early-staged esophageal cancers, gastric cancers, and colorectal cancers, and the like with EMR, ESD or the like, or advanced intra-abdominal surgery with NOTES.

### DESCRIPTION OF NUMERALS

100, 200 Endoscope overtube
110, 210 First insertion passage
115 Wall
116 Distal end
117 Proximal end
120, 220 Second insertion passage
125, 225 Opening

1-5. (canceled)

6. An endoscope overtube having a first insertion passage into which an endoscope can be inserted and having a substantially cylindrical shape, comprising
a second insertion passage into which a treatment instrument can be inserted from an opening at a proximal end thereof and from which the treatment instrument can be protruded from an opening at a distal end thereof, and through which a lubricant can be supplied, in a wall constituting the first insertion passage,
wherein the second insertion passage has, on a side face thereof, an opening which passes through a surface of the overtube.

7. The endoscope overtube of claim 6, wherein the second insertion passage is substantially parallel to the first insertion passage.

8. The endoscope overtube of claim 7, wherein 2 to 5 openings are provided on the side face along a major axis direction.

9. The endoscope overtube of claim 6, wherein a diameter of the opening on the side face is smaller than a diameter of the second insertion passage.

10. The endoscope overtube of claim 9, wherein diameters of the openings on the side face increase from the proximal end toward the distal end of the second insertion passage.

11. The endoscope overtube of claim 8, wherein intervals between the openings on the side face, and between the openings on the side face and the proximal end and the distal end of the second insertion passage are equal.

12. The endoscope overtube of claim 7, wherein a diameter of the opening on the side face is smaller than a diameter of the second insertion passage.

13. The endoscope overtube of claim 12, wherein diameters of the openings on the side face increase from the proximal end toward the distal end of the second insertion passage.

14. The endoscope overtube of claim 8, wherein a diameter of the opening on the side face is smaller than a diameter of the second insertion passage.

15. The endoscope overtube of claim 14, wherein diameters of the openings on the side face increase from the proximal end toward the distal end of the second insertion passage.

16. The endoscope overtube of claim 9, wherein intervals between the openings on the side face, and between the openings on the side face and the proximal end and the distal end of the second insertion passage are equal.

17. The endoscope overtube of claim 10, wherein intervals between the openings on the side face, and between the openings on the side face and the proximal end and the distal end of the second insertion passage are equal.
